(19) United States
(12) Patent Application Publication
(10) Pub. No.: US 2023/0410308 A1
(43) Pub. Date: Dec. 21, 2023

(54) DETECTION OF FOREIGN OBJECTS IN INTRAOPERATIVE IMAGES

(71) Applicant: Brainlab AG, Munich (DE)

(72) Inventors: Ferdinand STORCH, Munich (DE); Martin ADAMSKI, Munich (DE)

(21) Appl. No.: 18/038,459

(22) PCT Filed: Jan. 12, 2021
(86) PCT No.: PCT/EP21/50488

§ 371 Date: May 24, 2023

(51) Int. Cl. G06T 7/00; G06T 17/00; G06T 7/11; A61B 1/00; G16H 70/20

(52) U.S. Cl. CPC G06T 7/0014; G06T 17/00; G06T 7/11; A61B 1/000094; A61B 1/000096; G16H 70/20; G06T 2207/30101; G06T 2207/10016; G06T 2207/10121; G06T 2207/20081; G06T 2207/20132; G06T 2207/20021; G06T 2207/20084; A61B 1/043

(57) ABSTRACT

The disclosed computer-implemented method of detecting at least one foreign object in one or more intraoperative images encompasses the provision and use of one or more intraoperative images, which are compared to expected image content. This also includes particularly the use of live intraoperative video data that are acquired and used in the computer-implemented method defined herein. The method further encompasses the creation, i.e. the calculation and or provision, of expected image content based on several different inputs. Such creation of expected image content can be based on e.g. data associated with the patient's body undergoing a medical procedure, parameters that are indicative of said medical procedure, and/or imaging parameters of the individual imaging device used to generate said one or more intraoperative images. In a further step, a comparison between the expected image content and the one or more acquired intraoperative images is conducted, preferably using an image and/or video analysis algorithm for analyzing the at least one acquired intraoperative image and for automatically detecting the at least one foreign object in the intraoperative image.

### FIELD OF THE INVENTION

The present invention relates to a computer-implemented method of detecting at least one foreign object in one or more intraoperative images, a corresponding computer program, a computer readable medium storing the computer program, as well as a medical image analysis system.

### TECHNICAL BACKGROUND

Live fluoroscopy is the main intraoperative imaging modality for endovascular surgery. The accumulated fluoroscopy time per intervention can exceed one hour. This causes significant radiation doses for the patient and the physician. Objects that were unintentionally placed in the beam path can cause additional scatter radiation as well as artefacts and should be avoided. Furthermore, it is not uncommon that the surgeon's hands enter the beam path—intentionally or unintentionally.

Currently the operator of the imaging device has to manually ensure that no unintended objects are placed in the beam path, that no unnecessary images are created and that the collimation is as small as reasonable possible.

Disadvantages of such state of the art scenarios are that the success in avoiding unnecessary radiation by e.g. the medical practitioner depends on experience and vigilance of the operator, and is prone to human errors. Moreover, incidents are not necessarily documented and only limited measures if an incident is detected can be taken. This all adds additional cognitive load to the physician.

The inventors of the present invention have thus, in the context of making the present invention, identified the need for detecting incidents faster, for applying measures faster, for apply adjustments automatically, for reliably issue warnings, and for automatically document incidents.

The present invention has thus the object to provide for an improved detection of foreign objects and/or unexpected content in intraoperative images.

Aspects of the present invention, examples and exemplary steps and their embodiments are disclosed in the following. Different exemplary features of the invention can be combined in accordance with the invention wherever technically expedient and feasible.

### EXEMPLARY SHORT DESCRIPTION OF THE INVENTION

In the following, a short description of the specific features of the present invention is given, which shall not be understood to limit the invention only to the features or a combination of the features described in this section.

Technical terms are used by their common sense. If a specific meaning is conveyed to certain terms, definitions of terms will be given in the following in the context of which the terms are used.

The disclosed computer-implemented method of detecting at least one foreign object in one or more intraoperative images encompasses the provision and use of one or more intraoperative images, which are compared to expected image content. This also includes particularly the use of live intraoperative video data that are acquired and used in the computer-implemented method defined herein. Moreover, said images can be generated by many different imaging modalities, i.e. imaging methods, as will be described hereinafter in detail.

The method further encompasses the creation, i.e. the calculation and or provision, of expected image content based on several different inputs. Such creation of expected image content can be based on e.g. data associated with the patient's body undergoing a medical procedure, parameters that are indicative of said medical procedure, and/or imaging parameters of the individual imaging device used to generate said one or more intraoperative images. In general, such an input used to generate said expected image content are data characterizing the patient and/or the medical procedure. Said data may be provided in an embodiment by e.g. imaging device parameters of an imaging device used for generating the acquired intraoperative image; patient information, like for example patient age, height, gender, BMI, known anatomical anomalies, and/or implants. Said data used for the creation of the expected image content may be in an embodiment the medical procedure information describing a nature and/or application of the medical procedure, which the patient was undergoing when the at least one intraoperative image was acquired. In addition, one or more previous preferably pre-operative images of the patient may be used to create said expected image content.

In a further step, a comparison between the expected image content and the one or more acquired intraoperative images is conducted, preferably using an image and/or video analysis algorithm for analyzing the at least one acquired intraoperative image and for automatically detecting the at least one foreign object in the intraoperative image.

Advantageously, the presented method allows subsequently, i.e. after the method detected the foreign object, to trigger a reaction such that the presence of said detected foreign object can be avoided in the future. For example, causing a warning to a user is a reaction the presented method can trigger. This reaction can be generates and/or carried out by the system described herein. The corresponding control signal may be generated and may be sent from the computer/processing unit/calculation unit, carrying out the presented method to, for example, a user interface. Moreover, other non-limiting examples of reactions that can be triggered by the presented method are adjusting and/or suggesting a collimation of an imaging device used for generating the acquired intraoperative image, adjusting/suggesting a position and/or acquisition direction of an imaging device used for generating the acquired intraoperative image, adjusting/suggesting X-ray acquisition parameters like e.g. exposure time, voltage, ampere, and image acquisition frequency, stopping the acquisition of intraoperative images, initiating a documentation of a detection result of the detection, and/or adjusting/suggesting one or more parameters of a robotic arm used during the intraoperative imaging. Also this will be explained in detail hereinafter.

In particular embodiments, during the calculation of the expected image content a synthetic image of the imaging modality, with which the acquired intraoperative image was generated, is calculated. Such a synthetic image can then be used as the expected image content, as set out herein before and hereinafter in detail.

In particular embodiments thereof, the creation of the synthetic image encompasses the use, creation and/or or acquisition of a synthetic patient model, like e.g. the Atlas of Brainlab, as will be explained in detail hereinafter. In general, a synthetic patient model used in the context of the present invention is to be understood as a virtual representation of at least a part of the patient's anatomy. Such a synthetic patient model may be more or less detailed, as will be appreciated by the skilled reader.

### GENERAL DESCRIPTION OF THE INVENTION

In this section, a description of the general features of the present invention is given for example by referring to possible embodiments of the invention.

According to a first aspect of the present invention, a computer-implemented method of detecting at least one foreign object in one or more intraoperative images is presented. The method comprises the steps of acquiring at least one intraoperative image of at least a part of a patient's body undergoing a medical procedure (step S1); calculating or providing expected image content of the acquired intraoperative image based on data characterizing the patient and/or the medical procedure (step S2); and comparing, in a calculative manner, the acquired intraoperative image with the calculated/provided expected image content (step S3) thereby automatically detecting the at least one foreign object (step S4) in the intraoperative image.

The “foreign object” that is automatically detected by the computer/processing unit carrying out the presented method, may be e.g. a medical instrument, hands and/or finger bones of the physician carrying out the medical procedure, which are shown in the acquired image. But also parts of a patient anatomy, like e.g. bones, implants, and/or tissue could be a “foreign object” in a scenario where it is simply not expected to be in the acquired intraoperative image. Thus, the term “foreign object” also explicitly covers in the context of the present invention a part of the patient's anatomy which is not expected in the intraoperative image in view of the present medical procedure and/or imaging procedure that the patient of the acquired intraoperative images is or was undergoing. This also covers, that no patient part at all is comprised in the acquired intraoperative images, since also this is an “unexpected image content” and would be detected as “foreign object”.

Thus, the presented method allows detecting abnormalities in images, preferably in fluoroscopic images, more preferably in one or more fluoroscopic live videos. Such abnormalities could be e.g. hands of the surgeon, robot/instruments in the beam path, an unexpected body region, a patient position not as expected for intervention, e.g. lateral instead of supine, that no patient at all is present, and/or that the patient moved, e.g. in comparison to previous scan/image. All these advantageous automatic detections can be made by each of the method, the program, i.e. the software, and the medical image analysis system of the present invention.

In case such foreign objects and/or unexpected content is/are detected, the method, the program, and the medical image analysis system of the present invention can react, as will be described in detail in the context of embodiments, in a way that makes it possible to reduce e.g. unnecessary x-ray-exposure for the patient and the personnel, potentially reduce contrast agent for the patient, improve quality of acquired image by e.g. changing to better orientation, reduce occlusions, etc. This may be achieved by, for example, giving an optical or acoustical warning, wherein the intensity of the warning can be dependent on severity of the found abnormally of the “foreign object/unexpected content. Exemplary alternatives are adjusting collimation or suggesting collimation of the used imaging device, e.g. to leave out surgeon hands, or adjusting the imaging device position. Moreover, the method, the program, and the medical image analysis system of the present invention may also adjust the image acquisition direction, and/or may adjust the x-ray acquisition parameters like e.g. exposure time, voltage, ampere, and image acquisition frequency. Alternatively or in combination stopping the acquisition of x-ray images completely and/or documenting of the incident is a measure the present invention can contain.

To cause such reaction, the method, the program, and the medical image analysis system of the present invention may generate/cause corresponding control signals that can be sent to e.g. the X-ray imaging device, where the x-ray acquisition parameters like e.g. exposure time, voltage, ampere, and image acquisition frequency, are then accordingly adapted.

Advantageously, with the present invention the detection of foreign objects in intraoperative images is more reliable and more accurate and does not depend anymore on experience and vigilance of the operator. It is also not prone to human errors and incidents can safely be documented. Moreover, the medical practitioner does not have additional cognitive load since the foreign object detection is taken over by the software/device. Thus, with the present invention, incidents can be detected faster, measures and/or reactions can be applied faster, adjustments can be applied automatically, warnings can be issued reliably and incidents can automatically be document.

As is appreciated by the skilled reader, in step S2 the provided method comprises not only “calculating expected image content”, but also the provision thereof. This alternative covers, for example, the embodiment where a look up table is used. In such a look up table, objects can be stored as entries that are and/or are not expected to be present in images of said medical procedure. In this particular embodiment the method also comprises the step of comparing the automatically detected at least one foreign object of the intraoperative image with said entries in the look up table, as will be explained in more detail hereinafter.

It should be noted that the invention does not involve, in particular comprise, or encompass an invasive step, which would represent a substantial physical interference with the body requiring professional medical expertise to be carried out and entailing a substantial health risk even when carried out with the required professional care and expertise. For example, the invention does not comprise a step of positioning a medical implant in order to fasten it to an anatomical structure or a step of fastening the medical implant to the anatomical structure or a step of preparing the anatomical structure for having the medical implant fastened to it. More particularly, the invention does not involve any surgical or therapeutic activity. The invention is instead directed to computer-implemented calculations, particularly automated image analysis, and preferably the generation of signals to trigger a reaction depending on the outcome of the detection of the “foreign object”, as was outlined before in detail and will be explained even more hereinafter. For this reason alone, no surgical or therapeutic activity and in particular, no surgical or therapeutic step is necessitated or implied by carrying out the invention.

Moreover, said intraoperative images can be of many different imaging modalities, i.e. imaging methods, as will described hereinafter in detail.

According to an embodiment of the present invention, the at least one intraoperative image originates from a live fluoroscopy video stream or is a live fluoroscopy video stream.

Live fluoroscopy is the main intraoperative imaging modality for endovascular surgery. Hence, the processing unit carrying out this computer-implemented method may receive and use the image data from the X-rays device that obtained real-time moving images of the interior of the patient. This type of medical imaging using a fluoroscope allows a physician to see the internal structure and function of a patient, so that the pumping action of the heart or the motion of swallowing, for example, can be watched. In its simplest form, a fluoroscope consists of an X-ray source and a fluorescent screen, between which a patient is placed. However, most fluoroscopes have included X-ray image intensifiers and cameras as well, to improve the image's visibility and make it available on a remote display screen.

Thus, as will be explained in more detail hereinafter, in an aspect of the present invention, a fluoroscope is provided together with the medical image analysis system that carries out the method to detect foreign objects and/or unexpected content in the fluoroscopy images of said fluoroscope.

According to an embodiment of the present invention, the at least one intraoperative image is a quasi-live fluoroscopy video stream, which is analysed later/retarded with respect to the real medical procedure that is shown in the video. In yet another embodiment, the intraoperative images are recorded videos and are analysed with the method presented herein to detect foreign objects and/or unexpected content in the video after the medical procedure is over. This can beneficially used to document foreign objects and/or unexpected content.

According to an embodiment of the present invention, the data characterizing the patient and/or the medical procedure are embodied as at least one of:
imaging device parameters of an imaging device used for generating the acquired intraoperative image;
patient information, preferably age, height, gender, BMI, known anatomical anomalies, and/or implants;
medical procedure information describing a nature and/or application of the medical procedure, which the patient was undergoing when the at least one intraoperative image was acquired; and
one or more previous, preferably pre-operative, images of the patient.

This embodiment details examples of the “data characterizing the patient and/or the medical procedure” used in step S2 of the presented method.

Note that with the knowledge about the medical procedure, the method can deduce which body part, anatomy of interest, and/or patient positioning, like e.g. lateral, supine, prone, is to be expected. Other examples of medical procedure information are the following. If one knows what medical procedure is performed, the method can determine, e.g. based on comparison data stored in the device applying the method or by getting access to external data, which anatomical region is to be expected in the images, e.g. in a hip surgery, a hip bone is expected and not finger bones. In case the device usage is provided as said medical procedure information, e.g. that it is an endovascular procedure, it is expected that one should see a guide wire or a balloon, but no big, metal instruments. An example of the patient positioning as medical procedure information is that the medical procedure is carried out in e.g. the supine position. Furthermore, the imaging modality, (rough) imaging angulation (e.g. Fluoroscopy in A-P-Projection), are examples of medical procedure information.

The method may also provide a dynamic awareness in that sense that is capable of detecting at which step of the medical procedure one is. The method may thus use information to calculate the “expected image content” in the form of knowledge about the order, timing of device usage, and imaging usage. For example, no scissors should be detected in the patient's body in a final control image, and no imaging should start before the sheath is in place.

According to an embodiment of the present invention, the intraoperative image was generated with an imaging device of a first imaging modality, wherein the step of calculating expected image content (step S2) comprises the step of creating a synthetic image of the first imaging modality, which synthetic image represents the expected image content (step S5).

In other words, an artificial image is calculated by the device carrying out this method, which can then be used for the comparison with the real, preferably live intraoperative image which the device received.

This synthetic image can be calculated e.g. based on one or more imaging device parameters of the imaging device that generated the acquired intraoperative images, previous images, medical procedure information as was outlined just before, and/or based on patient information, like e.g. age, height, gender, BMI, known anatomical anomalies, and/or implants.

According to an embodiment of the present invention, the step of creating the synthetic image (step S5) further comprises the step of creating or acquiring a synthetic patient model (step S5a), adjusting the synthetic patient model based on patient information, preferably age, height, gender, BMI, known anatomical anomalies, and/or implants, and/or based on intraoperative image data (step S5b), and using the adjusted synthetic patient model in the creation of the synthetic image (step S5c).

The use of intraoperative image data for adjusting the synthetic patient model may be of particular advantage. If e.g. already one fluoroscopy image was generated, one could adapt the synthetic patient model such that the contour of the model fits and matches with the contour of said fluoroscopy image.

In general, the synthetic patient model used in the context of this embodiment shall be understood as a virtual representation of at least a part of the patient's anatomy. Such a synthetic patient model may be more or less detailed, as will be appreciated by the skilled reader. One non-limiting example of such a synthetic patient model is the Atlas of Brainlab. Such a synthetic patient model used in the context of this embodiment will now be described in the context of the Atlas, to which this embodiment is, however, explicitly not limited

Preferably, atlas data is acquired which describes (for example defines, more particularly represents and/or is) a general three-dimensional shape of the anatomical body part. The atlas data therefore represents an atlas of the anatomical body part. An atlas typically consists of a plurality of generic models of objects, wherein the generic models of the objects together form a complex structure. For example, the atlas constitutes a statistical model of a patient's body (for example, a part of the body) which has been generated from anatomic information gathered from a plurality of human bodies, for example from medical image data containing images of such human bodies. In principle, the atlas data therefore represents the result of a statistical analysis of such medical image data for a plurality of human bodies. This result can be output as an image—the atlas data therefore contains or is comparable to medical image data. Such a comparison can be carried out for example by applying an image fusion algorithm, which conducts an image fusion between the atlas data and the medical image data. The result of the comparison can be a measure of similarity between the atlas data and the medical image data. The atlas data comprises image information (for example, positional image information) which can be matched (for example by applying an elastic or rigid image fusion algorithm) for example to image information (for example, positional image information) contained in medical image data so as to for example compare the atlas data to the medical image data in order to determine the position of anatomical structures in the medical image data which correspond to anatomical structures defined by the atlas data.

The human bodies, the anatomy of which serves as an input for generating the atlas data, advantageously share a common feature such as at least one of gender, age, ethnicity, body measurements (e.g. size and/or mass) and pathologic state. The anatomic information describes for example the anatomy of the human bodies and is extracted for example from medical image information about the human bodies. The atlas of a femur, for example, can comprise the head, the neck, the body, the greater trochanter, the lesser trochanter and the lower extremity as objects, which together make up the complete structure. The atlas of a brain, for example, can comprise the telencephalon, the cerebellum, the diencephalon, the pons, the mesencephalon and the medulla as the objects, which together make up the complex structure. One application of such an atlas is in the segmentation of medical images, in which the atlas is matched to medical image data, and the image data are compared with the matched atlas in order to assign a point (a pixel or voxel) of the image data to an object of the matched atlas, thereby segmenting the image data into objects.

According to an embodiment of the present invention, the at least one intraoperative image is a 2D image, preferably a 2D fluoroscopy image. Moreover, wherein the step using the adjusted synthetic patient model in the creation of the synthetic image (step S5c) further comprises the steps of deriving a 3D image from the synthetic patient model, and deriving the synthetic image from the 3D image by calculating a Digitally Reconstructed Radiograph (DRR) thereby using imaging device parameters of the imaging device, which generated the 2D fluoroscopy image.

In case the synthetic image, which is adapted to the individual patient, is a CT in 3D, and the real, intraoperative image is a 2D fluoroscopy image, no direct comparison is possible. Thus, 2D fluoroscopy image has to be derived from the 3D CT, which can be done with a Digitally Reconstructed Radiograph. A projection of X-rays from a virtual X-ray source through the 3D CT is simulated, in which the angulation of the C-arm is used. In this embodiment, the method thus would entail the step or reading out the C-arm angulation data from the C-arm and using them in calculating the DRR.

According to an embodiment of the present invention, the step of creating the synthetic image (step S5) further comprises the step of virtually placing and/or orienting the adjusted synthetic patient model relative to the imaging device of the first imaging modality based on medical procedure information.

According to an embodiment of the present invention, the method further comprises the steps of segmenting anatomical structures in the synthetic image, segmenting anatomical structures in the acquired intraoperative image, and comparing the segmented images for detecting the at least one foreign object.

In this embodiment, the segmentation of objects in both images is introduced, i.e. in the synthetic and the real image. In digital image processing, image segmentation is the process of partitioning a digital image into multiple segments (sets of pixels, also known as image objects). The goal of segmentation is to simplify and/or change the representation of an image into something that is more meaningful and easier to analyze. Image segmentation is typically used to locate objects and boundaries (lines, curves, etc.) in images. More precisely, image segmentation is the process of assigning a label to every pixel in an image such that pixels with the same label share certain characteristics. The result of image segmentation is a set of segments that collectively cover the entire image, or a set of contours extracted from the image. Each of the pixels in a region are similar with respect to some characteristic or computed property, such as color, intensity, or texture. Adjacent regions are significantly different with respect to the same characteristic.

According to an embodiment of the present invention, the method further comprises the step of cropping the synthetic image based on positional information of the imaging device of the first imaging modality, a field of view of the imaging device of the first imaging modality, and/or medical procedure information.

This embodiment defines the step of cropping the image. Cropping is understood as the removal of unwanted outer areas from a photographic or illustrated image. The process usually consists of the removal of some of the peripheral areas of an image to remove extraneous content from the picture, to improve its framing, to change the aspect ratio, or to accentuate or isolate the subject matter from its background.

For example, the resulting DRR of the embodiment described herein before is cropped based on the C-arm position and/or field of view and/or procedural information, e.g. it is known, that the lesion is located on the left knee.

According to an embodiment of the present invention, the step of providing the expected image content of the acquired intraoperative image (step S2) comprises the steps of providing a look up table, in which objects are stored as entries that are and/or are not expected to be present in images of said medical procedure, and comparing the automatically detected at least one foreign object of the intraoperative image with said entries in the look up table.

In this embodiment, the comparison of objects in the actual image with objects in a generic look-up table is comprised, where entries like e.g. known implants, devices, etc. are stored. Thus, in this embodiment no synthetic image is required.

According to an embodiment of the present invention, the step of comparing, in a calculative manner, the acquired intraoperative image with the calculated/provided expected image content (step S3) comprises using an image and/or video analysis algorithm for analyzing the at least one acquired intraoperative image, preferably under consideration of parameters of an imaging device used to generate the acquired intraoperative image like e.g. C-arm settings, X-ray tube settings, and/or angulation.

The image and/or video analysis algorithms may comprise for example edge detection, object recognition, and/or motion detection (e.g. whether fingers are moving). The method may also use similarity measures like mutual information or correlations between the images that are compared. In another embodiment, histogram analysis is used to compare, in a calculative manner, the acquired intraoperative image with the calculated/provided expected image content.

According to an embodiment of the present invention, the video analysis algorithm uses machine learning, preferably a neural network.

In other words, the machine learning is facilitated by an artificial intelligence module, which is provided in the computer and/or processing unit and/or medical image analysis system, which carries out this embodiment of the presented method.

As is understood by the skilled reader, an artificial intelligence module is an entity that processes one or more inputs into one or more outputs by means of an internal processing chain that typically has a set of free parameters. The internal processing chain may be organized in interconnected layers that are traversed consecutively when proceeding from the input to the output.

Many artificial intelligence modules are organized to process an input having a high dimensionality into an output of a much lower dimensionality. For example, an image in HD resolution of 1920×1080 pixels lives in a space having a 1920×1080=2,073,600 dimensions. A common job for an artificial intelligence module is to classify images into one or more categories based on, for example, whether they contain certain objects. The output may then, for example, give, for each of the to-be-detected objects, a probability that the object is present in the input image. This output lives in a space having as many dimensions as there are to-be-detected objects. Typically, there are on the order of a few hundred or a few thousand to-be-detected objects.

Such a module is termed “intelligent” because it is capable of being “trained.” The module may be trained using records of training data. A record of training data comprises training input data and corresponding training output data. The training output data of a record of training data is the result that is expected to be produced by the module when being given the training input data of the same record of training data as input. The deviation between this expected result and the actual result produced by the module is observed and rated by means of a “loss function”. This loss function is used as a feedback for adjusting the parameters of the internal processing chain of the module. For example, the parameters may be adjusted with the optimization goal of minimizing the values of the loss function that result when all training input data is fed into the module and the outcome is compared with the corresponding training output data.

The result of this training is that given a relatively small number of records of training data as “ground truth”, the module is enabled to perform its job, e.g., the classification of images as to which objects they contain, well for a number of records of input data that higher by many orders of magnitude. For example, a set of about 100,000 training images that has been “labelled” with the ground truth of which objects are present in each image may be sufficient to train the module so that it can then recognize these objects in all possible input images, which may, e.g., be over 530 million images at a resolution of 1920×1080 pixels and a color depth of 8 bits.

Moreover, a neural network is a prime example of an internal processing chain of an artificial intelligence module. It consists of a plurality of layers, wherein each layer comprises one or more neurons. Neurons between adjacent layers are linked in that the outputs of neurons of a first layer are the inputs of one or more neurons in an adjacent second layer. Each such link is given a “weight” with which the corresponding input goes into an “activation function” that gives the output of the neuron as a function of its inputs. The activation function is typically a nonlinear function of its inputs. For example, the activation function may comprise a “pre-activation function” that is a weighted sum or other linear function of its inputs, and a thresholding function or other nonlinear function that produces the final output of the neuron from the value of the pre-activation function.

A convolutional neural network is a neural network that comprises “convolutional layers”. In a “convolutional layer”, the output of neurons is obtained by applying a convolution kernel to the inputs of these neurons. This greatly reduces the dimensionality of the data. Convolutional neural networks are frequently used in image processing.

A generative adversarial network is a combination of two neural networks termed “generator” and “discriminator”. Such a network is used to artificially produce records of data that are indistinguishable from records taken from a given set of training records of data. The generator network is trained with the goal of creating, from an input record with random data, an output record that is indistinguishable from the records in the set of training records. I.e., given that output record alone, it cannot be distinguished whether it has been produced by the generator or whether it is contained in the set of training records. The discriminator, in turn, is specifically trained to classify given records of data as to whether they are likely “real” training records or “fake” records produced by the generator. The generator and the discriminator thus compete against each other.

For example, a generative adversarial network may be used to create photorealistic images that are indistinguishable from a set of training images. From a limited number of training images obtained, e.g., by medical imaging, a near-infinite number of fake images that can pass for such medical images can be generated. A prime application of this is the production of training data for other artificial intelligence modules, e.g., modules that are to be trained to classify whether certain features or objects are present in medical images.

According to an embodiment of the present invention, the method further comprises
automatically generating, based on a detection result of step S4, a control signal, and wherein the control signal is configured for:
causing a warning to a user,
adjusting/suggesting a collimation of an imaging device used for generating the acquired intraoperative image,
adjusting/suggesting a position and/or acquisition direction of an imaging device used for generating the acquired intraoperative image,
adjusting/suggesting X-ray acquisition parameters like e.g. exposure time, voltage, ampere, and image acquisition frequency,
stopping the acquisition of intraoperative images,
initiating a documentation of a detection result of the detection of step S4, and/or
adjusting/suggesting one or more parameters of a robotic arm used during the intraoperative imaging.

In this embodiment, possibilities of a reaction, that is triggered by the method or by the device carrying out this method after the foreign object has automatically been detected, are detailed. Said “control signal” can be sent from the device, which carries out the method of the present invention to another device, like e.g. the X-ray device generating the 2D fluoroscopy images, or to a display to alert the user or the medical practitioner.

According to an embodiment of the present invention, in case a body part of a medical practitioner is automatically detected in step S3 as the at least one foreign object in the intraoperative image, the method comprises the step of automatically calculating an X-ray dose, which the detected body part of the medical practitioner receives during the medical procedure.

This embodiment automatically avoids that the medical practitioner receives an unintentional X-ray dose. For example, fluoroscopy is the main intraoperative imaging modality for endovascular surgery and the accumulated fluoroscopy time per intervention can exceed one hour. Thus, this causes significant radiation doses for the patient and the physician. Objects that were unintentionally placed in the beam path are now automatically detected with the present invention such that with the presented embodiment of the present invention it is instantly detected when the surgeon's hands enter the beam path—intentionally or unintentionally. In such a scenario, the method of this embodiment automatically determines the X-ray dose, which the detected body part of the medical practitioner receives during the medical procedure. Different values/parameters can be used in order to calculate said does, as will be explained hereinafter.

According to an embodiment of the present invention, the automatic calculation of the X-ray dose uses a power of the X-ray device, a surface area of the detected body part of the medical practitioner and an exposure time of the detected body part of the medical practitioner.

According to another aspect of the present invention, a program is presented, which, when running on a computer or when loaded onto a computer, causes the computer to perform the method steps of the method according to any one of the preceding claims.

This program can be seen as a computer program element and may be part of an existing computer program, but it can also be an entire program by itself. For example, the program may be used to update an already existing computer program to get to the present invention.

In this aspect, the invention is directed to a computer program which, when running on at least one processor (for example, a processor) of at least one computer (for example, a computer) or when loaded into at least one memory (for example, a memory) of at least one computer (for example, a computer), causes the at least one computer to perform the above-described method according to the first aspect. The invention may alternatively or additionally relate to a (physical, for example electrical, for example technically generated) signal wave, for example a digital signal wave, carrying information which represents the program, for example the aforementioned program, which for example comprises code means which are adapted to perform any or all of the steps of the method according to the first aspect. A computer program stored on a disc is a data file, and when the file is read out and transmitted it becomes a data stream for example in the form of a (physical, for example electrical, for example technically generated) signal. The signal can be implemented as the signal wave which is described herein. For example, the signal, for example the signal wave is constituted to be transmitted via a computer network, for example LAN, WLAN, WAN, for example the internet. The invention according to this aspect therefore may alternatively or additionally relate to a data stream representative of the aforementioned program.

According to another aspect of the present invention, a computer readable medium on which said program is stored, is presented.

Thus, this aspect of the invention is directed to a non-transitory computer-readable program storage medium on which the program according to the previous aspect is stored. The computer readable medium may be seen as a storage medium, such as for example, a USB stick, a CD, a DVD, a data storage device, a hard disk, or any other medium on which a program element as described above can be stored.

According to another aspect of the present invention, a medical image analysis system is presented, which comprises an image acquisition unit being configured for acquiring at least one intraoperative image of at least a part of a patient's body undergoing a medical procedure. Moreover, a processing unit is comprised, which is configured for calculating or providing expected image content of the acquired intraoperative image based on data characterizing the patient and/or the medical procedure. Moreover, the processing unit is configured for comparing, in a calculative manner, the acquired intraoperative image with the calculated/provided expected image content thereby automatically detecting the at least one foreign object in the intraoperative image.

According to an embodiment of the present invention, the medical image analysis system and the processing unit are configured to carry out any of methods described herein before and hereinafter.

According to another aspect of the present invention, a fluoroscope is provided together with the medical image analysis system. The fluoroscope comprise at least an X-ray source and a fluorescent screen. The intraoperative images acquired by the medical image analysis system are the fluoroscopy video images generated by the fluoroscope.

### Definitions

In this section, definitions for specific terminology used in this disclosure are offered which also form part of the present disclosure.

Computer Implemented Method

The method in accordance with the invention is for example a computer implemented method. For example, all the steps or merely some of the steps (i.e. less than the total number of steps) of the method in accordance with the invention can be executed by a computer (for example, at least one computer). An embodiment of the computer implemented method is a use of the computer for performing a data processing method. An embodiment of the computer implemented method is a method concerning the operation of the computer such that the computer is operated to perform one, more or all steps of the method.

The computer for example comprises at least one processor and for example at least one memory in order to (technically) process the data, for example electronically and/or optically. The processor being for example made of a substance or composition which is a semiconductor, for example at least partly n- and/or p-doped semiconductor, for example at least one of II-, III-, IV-, V-, VI-semiconductor material, for example (doped) silicon and/or gallium arsenide. The calculating or determining steps described are for example performed by a computer. Determining steps or calculating steps are for example steps of determining data within the framework of the technical method, for example within the framework of a program. A computer is for example any kind of data processing device, for example electronic data processing device. A computer can be a device which is generally thought of as such, for example desktop PCs, notebooks, netbooks, etc., but can also be any programmable apparatus, such as for example a mobile phone or an embedded processor. A computer can for example comprise a system (network) of “sub-computers”, wherein each sub-computer represents a computer in its own right. The term “computer” includes a cloud computer, for example a cloud server. The term “cloud computer” includes a cloud computer system which for example comprises a system of at least one cloud computer and for example a plurality of operatively interconnected cloud computers such as a server farm. Such a cloud computer is preferably connected to a wide area network such as the world wide web (WWW) and located in a so-called cloud of computers which are all connected to the world wide web. Such an infrastructure is used for “cloud computing”, which describes computation, software, data access and storage services which do not require the end user to know the physical location and/or configuration of the computer delivering a specific service. For example, the term “cloud” is used in this respect as a metaphor for the Internet (world wide web). For example, the cloud provides computing infrastructure as a service (IaaS). The cloud computer can function as a virtual host for an operating system and/or data processing application which is used to execute the method of the invention. The cloud computer is for example an elastic compute cloud (EC2) as provided by Amazon Web Services™. A computer for example comprises interfaces in order to receive or output data and/or perform an analogue-to-digital conversion. The data are for example data which represent physical properties and/or which are generated from technical signals. The technical signals are for example generated by means of (technical) detection devices (such as for example devices for detecting marker devices) and/or (technical) analytical devices (such as for example devices for performing (medical) imaging methods), wherein the technical signals are for example electrical or optical signals. The technical signals for example represent the data received or outputted by the computer. The computer is preferably operatively coupled to a display device which allows information outputted by the computer to be displayed, for example to a user. One example of a display device is a virtual reality device or an augmented reality device (also referred to as virtual reality glasses or augmented reality glasses) which can be used as “goggles” for navigating. A specific example of such augmented reality glasses is Google Glass (a trademark of Google, Inc.). An augmented reality device or a virtual reality device can be used both to input information into the computer by user interaction and to display information outputted by the computer. Another example of a display device would be a standard computer monitor comprising for example a liquid crystal display operatively coupled to the computer for receiving display control data from the computer for generating signals used to display image information content on the display device. A specific embodiment of such a computer monitor is a digital lightbox. An example of such a digital lightbox is Buzz®, a product of Brainlab AG. The monitor may also be the monitor of a portable, for example handheld, device such as a smart phone or personal digital assistant or digital media player.

The invention also relates to a program which, when running on a computer, causes the computer to perform one or more or all of the method steps described herein and/or to a program storage medium on which the program is stored (in particular in a non-transitory form) and/or to a computer comprising said program storage medium and/or to a (physical, for example electrical, for example technically generated) signal wave, for example a digital signal wave, carrying information which represents the program, for example the aforementioned program, which for example comprises code means which are adapted to perform any or all of the method steps described herein.

Within the framework of the invention, computer program elements can be embodied by hardware and/or software (this includes firmware, resident software, micro-code, etc.). Within the framework of the invention, computer program elements can take the form of a computer program product which can be embodied by a computer-usable, for example computer-readable data storage medium comprising computer-usable, for example computer-readable program instructions, “code” or a “computer program” embodied in said data storage medium for use on or in connection with the instruction-executing system. Such a system can be a computer; a computer can be a data processing device comprising means for executing the computer program elements and/or the program in accordance with the invention, for example a data processing device comprising a digital processor (central processing unit or CPU) which executes the computer program elements, and optionally a volatile memory (for example a random access memory or RAM) for storing data used for and/or produced by executing the computer program elements. Within the framework of the present invention, a computer-usable, for example computer-readable data storage medium can be any data storage medium which can include, store, communicate, propagate or transport the program for use on or in connection with the instruction-executing system, apparatus or device. The computer-usable, for example computer-readable data storage medium can for example be, but is not limited to, an electronic, magnetic, optical, electromagnetic, infrared or semiconductor system, apparatus or device or a medium of propagation such as for example the Internet. The computer-usable or computer-readable data storage medium could even for example be paper or another suitable medium onto which the program is printed, since the program could be electronically captured, for example by optically scanning the paper or other suitable medium, and then compiled, interpreted or otherwise processed in a suitable manner. The data storage medium is preferably a non-volatile data storage medium. The computer program product and any software and/or hardware described here form the various means for performing the functions of the invention in the example embodiments. The computer and/or data processing device can for example include a guidance information device which includes means for outputting guidance information. The guidance information can be outputted, for example to a user, visually by a visual indicating means (for example, a monitor and/or a lamp) and/or acoustically by an acoustic indicating means (for example, a loudspeaker and/or a digital speech output device) and/or tactilely by a tactile indicating means (for example, a vibrating element or a vibration element incorporated into an instrument). For the purpose of this document, a computer is a technical computer which for example comprises technical, for example tangible components, for example mechanical and/or electronic components. Any device mentioned as such in this document is a technical and for example tangible device.

Acquiring Data/an Image

The expression “acquiring data” and/or “acquiring an image” (which will be used herein synonymously) for example encompasses (within the framework of a computer implemented method) the scenario in which the data/image data are determined by the computer implemented method or program. Determining data for example encompasses measuring physical quantities and transforming the measured values into data, for example digital data, and/or computing (and e.g. outputting) the data by means of a computer and for example within the framework of the method in accordance with the invention. The meaning of “acquiring data”/“acquiring an image” also for example encompasses the scenario in which the data are received or retrieved by (e.g. input to) the computer implemented method or program, for example from another program, a previous method step or a data storage medium, for example for further processing by the computer implemented method or program. Generation of the data to be acquired may but need not be part of the method in accordance with the invention. The expression “acquiring data” can therefore also for example mean waiting to receive data and/or receiving the data. The received data can for example be inputted via an interface. The expression “acquiring data” can also mean that the computer implemented method or program performs steps in order to (actively) receive or retrieve the data from a data source, for instance a data storage medium (such as for example a ROM, RAM, database, hard drive, etc.), or via the interface (for instance, from another computer or a network). The data acquired by the disclosed method or device, respectively, may be acquired from a database located in a data storage device which is operably to a computer for data transfer between the database and the computer, for example from the database to the computer. The computer acquires the data for use as an input for steps of determining data. The determined data can be output again to the same or another database to be stored for later use. The database or database used for implementing the disclosed method can be located on network data storage device or a network server (for example, a cloud data storage device or a cloud server) or a local data storage device (such as a mass storage device operably connected to at least one computer executing the disclosed method). The data can be made “ready for use” by performing an additional step before the acquiring step. In accordance with this additional step, the data are generated in order to be acquired. The data are for example detected or captured (for example by an analytical device). Alternatively or additionally, the data are inputted in accordance with the additional step, for instance via interfaces. The data generated can for example be inputted (for instance into the computer). In accordance with the additional step (which precedes the acquiring step), the data can also be provided by performing the additional step of storing the data in a data storage medium (such as for example a ROM, RAM, CD and/or hard drive), such that they are ready for use within the framework of the method or program in accordance with the invention. The step of “acquiring data” can therefore also involve commanding a device to obtain and/or provide the data to be acquired. In particular, the acquiring step does not involve an invasive step which would represent a substantial physical interference with the body, requiring professional medical expertise to be carried out and entailing a substantial health risk even when carried out with the required professional care and expertise. In particular, the step of acquiring data, for example determining data, does not involve a surgical step and in particular does not involve a step of treating a human or animal body using surgery or therapy. In order to distinguish the different data used by the present method, the data are denoted (i.e. referred to) as “XY data” and the like and are defined in terms of the information which they describe, which is then preferably referred to as “XY information” and the like.

Imaging Methods

In the field of medicine, imaging methods (also called imaging modalities and/or medical imaging modalities) are used to generate image data (for example, two-dimensional or three-dimensional image data) of anatomical structures (such as soft tissues, bones, organs, etc.) of the human body. The term “medical imaging methods” is understood to mean (advantageously apparatus-based) imaging methods (for example so-called medical imaging modalities and/or radiological imaging methods) such as for instance computed tomography (CT) and cone beam computed tomography (CBCT, such as volumetric CBCT), x-ray tomography, magnetic resonance tomography (MRT or MRI), conventional x-ray, sonography and/or ultrasound examinations, and positron emission tomography. For example, the medical imaging methods are performed by the analytical devices. Examples for medical imaging modalities applied by medical imaging methods are: X-ray radiography, magnetic resonance imaging, medical ultrasonography or ultrasound, endoscopy, elastography, tactile imaging, thermography, medical photography and nuclear medicine functional imaging techniques as positron emission tomography (PET) and Single-photon emission computed tomography (SPECT), as mentioned by Wikipedia.

The image data thus generated is also termed “medical imaging data”. Analytical devices for example are used to generate the image data in apparatus-based imaging methods. The imaging methods are for example used for medical diagnostics, to analyse the anatomical body in order to generate images which are described by the image data. The imaging methods are also for example used to detect pathological changes in the human body. However, some of the changes in the anatomical structure, such as the pathological changes in the structures (tissue), may not be detectable and for example may not be visible in the images generated by the imaging methods. A tumour represents an example of a change in an anatomical structure. If the tumour grows, it may then be said to represent an expanded anatomical structure. This expanded anatomical structure may not be detectable; for example, only a part of the expanded anatomical structure may be detectable. Primary/high-grade brain tumours are for example usually visible on MRI scans when contrast agents are used to infiltrate the tumour. MRI scans represent an example of an imaging method. In the case of MRI scans of such brain tumours, the signal enhancement in the MRI images (due to the contrast agents infiltrating the tumour) is considered to represent the solid tumour mass. Thus, the tumour is detectable and for example discernible in the image generated by the imaging method. In addition to these tumours, referred to as “enhancing” tumours, it is thought that approximately 10% of brain tumours are not discernible on a scan and are for example not visible to a user looking at the images generated by the imaging method.

### BRIEF DESCRIPTION OF THE DRAWINGS

In the following, the invention is described with reference to the appended figures, which give background explanations and represent specific embodiments of the invention. The scope of the invention is however not limited to the specific features disclosed in the context of the figures, wherein

FIG. 1 shows a flow diagram of the computer-implemented method according to the present invention;

FIG. 2 shows a flow diagram of a computer-implemented method according to an embodiment of the present invention;

FIG. 3 schematically shows a fluoroscope with a medical image analysis system according to an exemplary embodiment of the present invention;

FIG. 4 schematically shows the creation of expected image content based on several different inputs according to an exemplary embodiment of the present invention.

### DESCRIPTION OF EMBODIMENTS

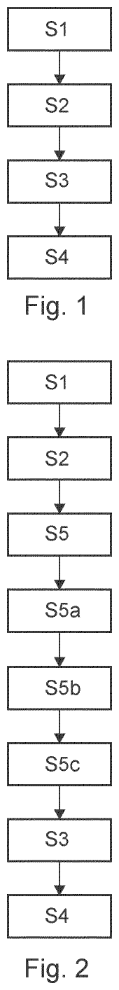

FIG. 1 schematically shows the computer-implemented method of detecting at least one foreign object in one or more intraoperative images and includes steps S1 to S4. In detail, the method comprises the steps of acquiring at least one intraoperative image of at least a part of a patient's body undergoing a medical procedure (step S1) and calculating or providing expected image content of the acquired intraoperative image based on data characterizing the patient and/or the medical procedure (step S2). Moreover, comparing, in a calculative manner, the acquired intraoperative image with the calculated/provided expected image content (step S3) is part of this method, thereby the method automatically detects the at least one foreign object (step S4) in the intraoperative image.

Advantageously, the presented method allows subsequently, after the method detected the foreign object, to trigger a reaction such that the presence of said detected foreign object can be avoided in the future. For example, causing a warning to a user is a reaction the presented method can trigger. The corresponding control signal may be generated and may be sent from the computer/processing unit/calculation unit, carrying out the presented method to, for example, a user interface. Moreover, other non-limiting examples of reactions that can be triggered by the presented method are adjusting and/or suggesting a collimation of an imaging device used for generating the acquired intraoperative image, adjusting and/or suggesting a position and/or acquisition direction of an imaging device used for generating the acquired intraoperative image, adjusting and/or suggesting X-ray acquisition parameters like e.g. exposure time, voltage, ampere, and image acquisition frequency, stopping the acquisition of intraoperative images, initiating a documentation of a detection result of the detection, and adjusting and/or suggesting one or more parameters of a robotic arm used during the intraoperative imaging.

Advantageously, with this method of FIG. 1 the detection of foreign objects in intraoperative images is more reliable and more accurate and does not depend anymore on experience and vigilance of the operator. It is also not prone to human errors and incidents can safely be documented. Moreover, the medical practitioner does not have additional cognitive load since the foreign object detection is taken over by the software/device. Thus, with the present invention, incidents can be detected faster, measures and/or reactions can be applied faster, adjustments can be applied automatically, warnings can be issued reliably and incidents can automatically be document. The “foreign object” that is automatically detected by the computer/processing unit carrying out the method of FIG. 1, may be e.g. a medical instrument, hands and/or finger bones of the physician carrying out the medical procedure, which are shown in the acquired image. But also parts of a patient anatomy, like e.g. bones, implants, and/or tissue could be a “foreign object” in a scenario where it is simply not expected to be in the acquired intraoperative image. Thus, the term “foreign object” also explicitly covers a part of the patient's anatomy which is not expected in the intraoperative image in view of the present medical procedure and/or imaging procedure that the patient of the acquired intraoperative images is or was undergoing. This also covers, that no patient part at all is comprised in the acquired intraoperative images, since also this is an “unexpected image content” and would be detected as “foreign object”.

FIG. 2 schematically shows a flow diagram of an embodiment of the computer-implemented detection method of the present invention. Regarding steps S1, S2, S3 and S4 it is referred to the previous description of the method of FIG. 1. In the method of FIG. 2 the intraoperative image was generated with an imaging device of a first imaging modality. In the step of calculating expected image content (i.e. step S2) the following step is comprised: creating a synthetic image of the first imaging modality, which synthetic image represents the expected image content, which is step S5. Moreover, the step of creating the synthetic image, i.e. step S5, further comprises the step of creating or acquiring a synthetic patient model in step S5a. In general, the synthetic patient model used in the context of this embodiment shall be understood as a virtual representation of at least a part of the patient's anatomy. Such a synthetic patient model may be more or less detailed, as will be appreciated by the skilled reader. One non-limiting example of such a synthetic patient model is the Atlas of Brainlab. Such a synthetic patient model can be used in the context of this embodiment. Said Atlas model was described herein before in detail.

Moreover, the step of creating the synthetic image, i.e. step S5, further comprises the step adjusting the synthetic patient model based on patient information, i.e. step S5b. In this embodiment, the method is configured to use any parameter of age, height, gender, BMI, known anatomical anomalies, and/or implants to adjust the patient model, i.e. step S5b. In addition or alternatively, intraoperative image data can be used for the adjustment of the synthetic patient model step of S5b. The use of intraoperative image data for adjusting the synthetic patient model in step S5b may be of particular advantage. If e.g. already one fluoroscopy image was generated, one could adapt the synthetic patient model such that the contour of the model fits and matches with the contour of said fluoroscopy image. The step of creating the synthetic image, i.e. step S5, further comprises the step S5c of using the adjusted synthetic patient model to create the synthetic image.

In the following, an even more detailed embodiment of the method described in FIG. 2 will be elucidated with the following method steps.
1) Create a synthetic image that reflects the expected image content. The following steps are exemplary and assume that the video stream is a fluoroscopic video stream.
a. Create/acquire a synthetic patient model that corresponds to the modality of the video stream (in this example it would be synthetic CT).
b. Adjust the synthetic patient model based on patient information (e.g. height, age, gender, BMI, known anatomical anomalies, implants, pre-operative imaging).
c. Virtually Place and orient the synthetic patient model relative to the imaging device according to medical procedure information (e.g. if a PAD procedure is performed: supine position)
d. Derive a synthetic DRR from the synthetic CT. The projection angulation is derived from the angulation of the actual C-arm that was determined from step c.
e. The resulting DRR is cropped based on the C-Arm position and/or FOV and/or procedural information (e.g. it is known, that the lesion is located on the left knee).
f. The resulting image is stored
g. Optional: anatomical structures in the image are segmented and stored
2) Compare synthetic image with actual image/video stream
   Option 1: direct comparison between the actual image and the image created in “f”.
   Option 2: comparison of structures that were segmented in step “g.” with structures that were segmented in the actual image

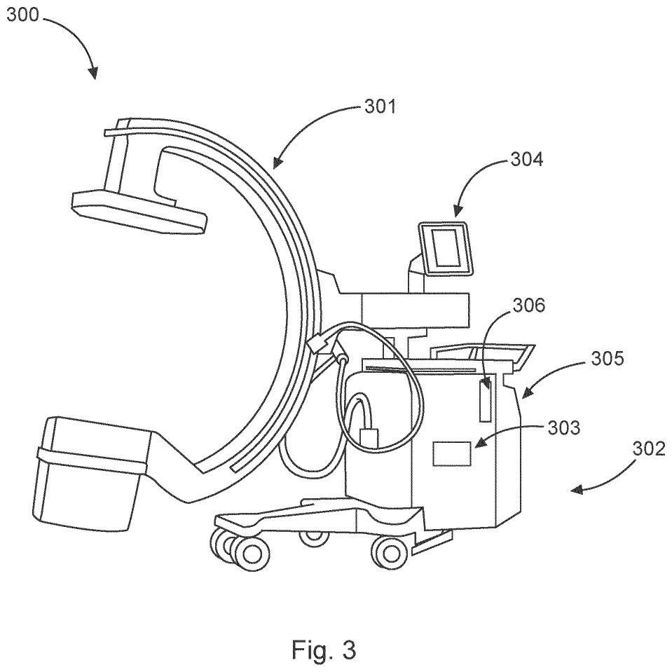

FIG. 3 schematically shows a fluoroscope 300 with a medical image analysis system 302 according to an exemplary embodiment of the present invention. The medical image analysis system 302 comprises a display 304, an image acquisition unit 306, which is configured for acquiring at least one intraoperative image generated by C-arm 301 and depicting at least a part of a patient's body undergoing a medical procedure. The medical image analysis system 302 further comprises a computer 305 with a processing unit 303, which is configured for calculating or providing expected image content of the acquired intraoperative image based on data characterizing the patient and/or the medical procedure. The processing unit 303 is also configured for comparing, in a calculative manner, the acquired intraoperative image with the calculated/provided expected image content thereby automatically detecting the at least one foreign object in the intraoperative image. Note that a system 302 shown in FIG. 3 in principle can be configured to carry out any of the computer-implemented methods mentioned herein.

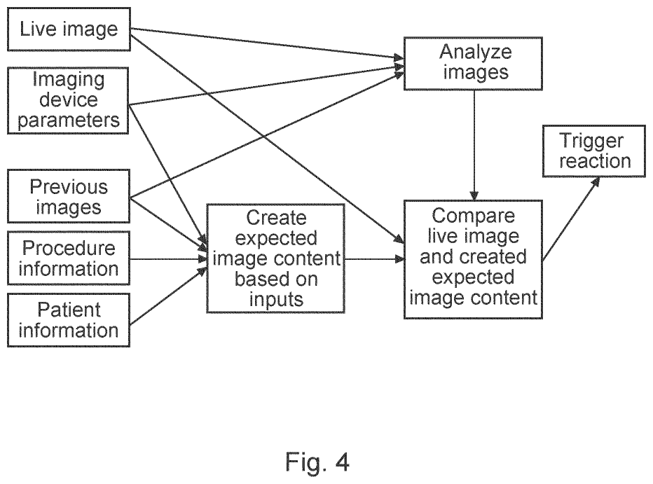

FIG. 4 schematically shows the creation of expected image content based on several different inputs according to an exemplary embodiment of the present invention. As can be gathered from FIG. 4, such creation of expected image content can be based on e.g. data associated with the patient's body undergoing a medical procedure, parameters that are indicative of said medical procedure, and/or imaging parameters of the individual imaging device used to generate said one or more intraoperative images. In general, such an input used to generate said expected image content are data characterizing the patient and/or the medical procedure. Said data may be provided in an embodiment by e.g. imaging device parameters of an imaging device used for generating the acquired intraoperative image; patient information, like for example patient age, height, gender, BMI, known anatomical anomalies, and/or implants. Said data used for the creation of the expected image content may be an embodiment the medical procedure information describing a nature and/or application of the medical procedure, which the patient was undergoing when the at least one intraoperative image was acquired. Also one or more previous, preferably pre-operative, images of the patient may be used to create said expected image content.

Advantageously, the detection of foreign objects in intraoperative images shown in FIG. 4 is more reliable and more accurate and does not depend anymore on experience and vigilance of the operator. It is also not prone to human errors and incidents can safely be documented. Moreover, the medical practitioner does not have additional cognitive load since the foreign object detection is taken over by the software/device. Thus, with the present invention, incidents can be detected faster, measures and/or reactions can be applied faster, adjustments can be applied automatically, warnings can be issued reliably and incidents can automatically be document.

Other variations to the disclosed embodiments can be understood and effected by those skilled in the art in practicing the claimed invention, from the study of the drawings, the disclosure, and the appended claims. In the claims the word “comprising” does not exclude other elements or steps and the indefinite article “a” or “an” does not exclude a plurality. A single processor or other unit may fulfil the functions of several items or steps recited in the claims. The mere fact that certain measures are recited in mutually different dependent claims does not indicate that a combination of these measures cannot be used to advantage. A computer program may be stored/distributed on a suitable medium such as an optical storage medium or a solid-state medium supplied together with or as part of other hardware, but may also be distributed in other forms, such as via the Internet or other wired or wireless telecommunication systems. Any reference signs in the claims should not be construed as limiting the scope of the claims.

1. A computer-implemented method of detecting at least one foreign object in one or more intraoperative images, the method comprising the steps:
acquiring at least one intraoperative image of at least a part of a patient's body undergoing a medical procedure;
calculating or providing expected image content of the acquired intraoperative image based on data characterizing the patient and/or the medical procedure; and
comparing, in a calculative manner, the acquired intraoperative image with the calculated/provided expected image content thereby automatically detecting the at least one foreign object in the intraoperative image.

2. The computer-implemented method according to claim 1, wherein the at least one intraoperative image is a live fluoroscopy video stream.

3. The computer-implemented method according to claim 1,
wherein the data characterizing the patient and/or the medical procedure are embodied as at least one of:
imaging device parameters of an imaging device used for generating the acquired intraoperative image;
patient information;
medical procedure information describing a nature and/or application of the medical procedure, which the patient was undergoing when the at least one intraoperative image was acquired; and
one or more previous images of the patient.

4. The computer-implemented method according to claim 1, wherein the intraoperative image was generated with an imaging device of a first imaging modality, wherein the step of calculating expected image content comprises:
creating a synthetic image of the first imaging modality, the synthetic image representing the expected image content.

5. The computer-implemented method according to claim 4, wherein the step of creating the synthetic image further comprises:
creating or acquiring a synthetic patient model,
adjusting the synthetic patient model based on patient information, and/or based on intraoperative image data, and
using the adjusted synthetic patient model in the creation of the synthetic image.

6. The computer-implemented method according to claim 5, wherein the at least one intraoperative image is a 2D image, wherein the step of using the adjusted synthetic patient model in the creation of the synthetic image further comprises:
deriving a 3D image from the synthetic patient model, and
deriving the synthetic image from the 3D image by calculating a Digitally Reconstructed Radiograph (DRR) thereby using imaging device parameters of the imaging device, which generated the 2D image.

7. The computer-implemented method according to claim 5, wherein the step of creating the synthetic image further comprises:
virtually placing and/or orienting the adjusted synthetic patient model relative to the imaging device of the first imaging modality based on medical procedure information.

8. The computer-implemented method according to claim 4, the method further comprising the steps:
segmenting anatomical structures in the synthetic image,
segmenting anatomical structures in the acquired intraoperative image, and
comparing the segmented images for detecting the at least one foreign object.

9. The computer-implemented method according to claim 4, the method further comprising the steps:
cropping the synthetic image based on positional information of the imaging device of the first imaging modality, a field of view of the imaging device of the first imaging modality, and/or medical procedure information.

10. The computer-implemented method according to claim 1, wherein the step of providing the expected image content of the acquired intraoperative image comprises:
providing a look up table, in which objects are stored as entries that are and/or are not expected to be present in images of the medical procedure, and
comparing the automatically detected at least one foreign object of the intraoperative image with the entries in the look up table.

11. The computer-implemented method according to claim 1, wherein the step of comparing, in a calculative manner, the acquired intraoperative image with the calculated/provided expected image content comprises:
using an image analysis algorithm and/or video analysis algorithm for analyzing the at least one acquired intraoperative image.

12. The computer-implemented method according to claim 11, wherein the video analysis algorithm uses machine learning.

13. The computer-implemented method according to claim 11, wherein the video analysis algorithm uses a histogram analysis.

14. The computer-implemented method according to claim 1, the method further comprising:
automatically generating, based on the detection of the at least one foreign object, a control signal, and wherein the control signal is configured for:
causing a warning to a user,
adjusting/suggesting a collimation of an imaging device used for generating the acquired intraoperative image,
adjusting/suggesting a position and/or acquisition direction of an imaging device used for generating the acquired intraoperative image,
adjusting/suggesting X-ray acquisition parameters,
stopping the acquisition of intraoperative images,
initiating a documentation of a detection result of the detection of the at least one foreign object, and/or
adjusting/suggesting one or more parameters of a robotic arm used during the intraoperative imaging.

15. The computer-implemented method according to claim 1, wherein in case a body part of a medical practitioner is automatically detected in comparing the acquired intraoperative image with the calculated/provided expected image content as the at least one foreign object in the intraoperative image, the method comprises the step of:
automatically calculating an X-ray dose, which the detected body part of the medical practitioner receives during the medical procedure.

16. The computer-implemented method according to claim 15, wherein the automatic calculation of the X-ray dose uses a power of the X-ray device, a surface area of the detected body part of the medical practitioner and an exposure time of the detected body part of the medical practitioner.

17. A non-transitory computer-readable storage medium storing a program, that when executed on at least one processor of a computer or when loaded onto the at least one processor of the computer, causes the computer to perform a method to detect at least one foreign object in one or more intraoperative images, the method comprising:
acquiring at least one intraoperative image of at least a part of a patient's body undergoing a medical procedure;
calculating or providing expected image content of the acquired intraoperative image based on data characterizing the patient and/or the medical procedure; and
comparing the acquired intraoperative image with the calculated/provided expected image content thereby automatically detecting the at least one foreign object in the intraoperative image.

18. (canceled)

19. A medical image analysis system comprising:
an image acquisition unit which is configured to acquire at least one intraoperative image of at least a part of a patient's body undergoing a medical procedure; and
a processing unit which is configured to:
calculate or provide expected image content of the acquired intraoperative image based on data characterizing the patient and/or the medical procedure; and
compare, in a calculative manner, the acquired intraoperative image with the calculated/provided expected image content thereby automatically detecting the at least one foreign object in the intraoperative image.

20. (canceled)

21. The medical image analysis system according to claim 19, wherein the at least one intraoperative image is a live fluoroscopy video stream.
